(19) United States
(12) Patent Application Publication
(10) Pub. No.: US 2021/0145708 A1
(43) Pub. Date: May 20, 2021

(54) WATER-IN-OIL COSMETIC COMPOSITION WITH EXCELLENT FLUIDITY AND STABILITY

(71) Applicant: LG HOUSEHOLD & HEALTH CARE LTD., Seoul (KR)

(72) Inventors: Jae-Sik HAN, Seoul (KR); Ho-Jung LEE, Seoul (KR); Sung-Soo KANG, Seoul (KR)

(73) Assignee: LG HOUSEHOLD & HEALTH CARE LTD., Seoul (KR)

(21) Appl. No.: 17/097,192

(22) Filed: Nov. 13, 2020

(30) Foreign Application Priority Data
Nov. 15, 2019 (KR) 10-2019-0146568
Mar. 11, 2020 (KR) 10-2020-0030276

(51) Int. Cl. A61K 8/06; A61K 8/86; A61K 8/891; A61K 8/895; A61K 8/92; A61K 8/37

(52) U.S. Cl. CPC A61K 8/064; A61K 8/86; A61K 8/891; A61K 8/895; A61K 2800/805; A61K 8/37; A61K 2800/412; A61K 2800/59; A61K 8/92

(57) ABSTRACT

The present invention relates to a water-in-oil cosmetic composition with excellent fluidity and stability, comprising a large amount of aqueous phase. The water-in-oil cosmetic composition of the present invention has excellent filling property, stability at high temperature and long term stability, by forming a desirable fluidity in the process of preparation at high temperature. Preferably, the water-in-oil cosmetic composition of the present invention may excellently exhibit a water-drop effect due to high content of aqueous phase.

### TECHNICAL FIELD

The present application claims the priority based on Korean Patent Application No. 10-2019-0146568 filed on Nov. 15, 2019 and the priority based on Korean Patent Application No. 10-2020-0030276 filed on Mar. 11, 2020, and the entire contents disclosed in the description and drawings of the corresponding applications incorporated in the present application. The present invention relates to a water-in-oil cosmetic composition with excellent fluidity, excellent high temperature stability and long term stability, comprising a large amount of aqueous phase.

### BACKGROUND ART

Recently, as the demand for makeup products that additionally provide skin care functions increases, products such as essence fact that provide refreshing, moisturizing or excellent cosmetic effects are spotlighted. The essence pact has shape of conventional pack-type cosmetic products such as skin cover products or two-way cake products including wax, emollient oil or powder ingredients. Therefore, the essence pact provides a simple makeup method, and can maintain makeup effect for a long time, because ingredients such as wax provide skin-covering effects by forming film-like layer onto skin. The film-like layer has excellent adhesion to the skin. In addition, the essence pact has a characteristic that water drops are produced by rubbing makeup surface on skin, and it makes an instant moisturizing effect. This water-drop forming phenomenon is known as a water-drop effect, and is highly useful in marketing because users can visually recognize moisturizing or cooling effects, etc.

Foundation products in a balm type are prepared by mixing various kinds of resin, wax or polar oil in order to provide necessary hardness to the products. Adding these ingredients may generally provide long lasting and higher covering effect because of excellent adhesion to skin and forming film-like layer onto skin. However, it makes increase in viscosity and heavy feeling in use.

Conventionally, in order to provide refreshing and moisturizing effects in use, the balm type products in water-in-oil formulation that contains a wax in the external phase (oil phase) and stabilizes aqueous ingredients in the internal phase (aqueous phase) have been developed. However, as the proportion of the inner phase increases, the viscosity increases accordingly. The increasing the proportion of inner phase reduces the fluidity of emulsion, and makes difficulties in emulsifying, filling or molding. Accordingly, difficulties occurred in the manufacturing process and productivity was reduced.

To overcome these problems, attempts have been made to control the size or fluidity of emulsified particles by reducing the amount of an emulsifier. However, this results in unstable emulsion and agglomeration of particles at the time of producing using high temperature. In addition, it makes the product unstable, and deteriorates feeling in use. Accordingly, conventional products could not provide sufficient moisturizing effect, because of the failure in including higher volume of internal aqueous phase in a water-in-oil cosmetic composition.

### DISCLOSURE

### Technical Problem

A problem to be solved by the present invention is to provide a cosmetic composition which has excellent filling ability, stability at high temperature and long term stability, by forming sufficient fluidity (viscosity) at high temperature even if the ratio of internal aqueous phase increases in a water-in-oil cosmetic composition.

In addition, another problem to be solved by the present invention is to provide a cosmetic composition that preferably exhibits a water-drop effect.

### Technical Solution

In order to overcome the above-mentioned problems of the prior art, the present invention provides a water-in-oil cosmetic composition comprising a combination of a surfactant comprising one hydrophilic group and two hydrophobic groups and a silicon crosspolymer comprising a hydrophilic group. The present inventors found that the cosmetic composition comprising the above combination forms particles over a particular size homogeneously in an emulsified state; the cosmetic composition has excellent emulsion fluidity in the process of preparation at high temperature; the cosmetic composition has the excellent filling property without agglomeration between particles; and the cosmetic composition has the excellent long term stability without causing sweating phenomenon. The present invention was completed through the above surprising findings of the inventers

In addition, the present inventors found that the cosmetic composition comprising the above combination could preferably provide the water-drop effect.

In one embodiment, the present invention provides a water-in-oil cosmetic composition comprising a surfactant comprising one hydrophilic group and two hydrophobic groups, a silicon crosspolymer comprising a hydrophilic group and wax. Wherein the wax may be comprised in an amount of 2 to 10% by weight based on the total weight of the composition. The water-in-oil cosmetic composition may contain a large amount of aqueous phase by comprising a surfactant comprising one hydrophilic group and two hydrophobic groups. Accordingly, the cosmetic composition improves the fluidity by reducing the interaction between the emulsified particles, and provides an immediate moisturizing feeling when applied to skin. In addition, the cosmetic composition comprises a silicon crosspolymer comprising a hydrophilic group, so that the binding portion of hydrophilic group surrounds emulsified particles in aqueous phase in a molten state at high temperature. In addition, the silicone, preferably, the main chain of dimethicone forms a particular structure and prevents emulsified particles from being merged with each other, thereby improving stability at high temperature and long-term stability of the composition.

In one embodiment, in the surfactant comprising one hydrophilic group and two hydrophobic groups, the one hydrophilic group is a polar head. A chain type saturated hydrocarbons having 16 to 24 carbon atoms or derivatives thereof are extended to both sides of the polar head. The hydrophilic group may be one or more selected from the group consisting of polyethylene glycol, polyglycerin, and polypropylene glycol, and the like to form a polar head. Preferably, the hydrophilic group may be polyethylene glycol to form a polar head. The surfactant comprising one hydrophilic group and two hydrophobic groups may be one or more selected from the group consisting of PEG-30 dipolyhydroxystearate, PEG-20 dipolyhydroxymyristate, PEG-40 dipolyhydroxypalmitate, PEG-4 trimethylolpropanedistearate, PEG-4 glyceryldistearate, polyglyceryl-2 diisostearate, polyglyceryl-diisostearate, polyglyceryl-3 distearate, PEG-6 distearate, PEG-6 diisostearate, PEG-8 distearate and PEG-8 diisostearate, and the like, but not limited thereto. Preferably, the surfactant comprising one hydrophilic group and two hydrophobic groups may be PEG-30 dipolyhydroxystearate. In this case, the fluidity (viscosity), stability at high temperature and long term stability of the water-in-oil cosmetic composition are significantly improved.

In one embodiment, the silicon crosspolymer comprising a hydrophilic group may be a crosspolymer which has dimethicone as a main chain. The silicon crosspolymer comprises a bond of one or more hydrophilic group selected from the group consisting of polyethylene glycol, polyglycerin and polypropylene glycol, and the like. The silicon crosspolymer comprising a hydrophilic group may be one or more selected from the group consisting of PEG-10 dimethicone/vinyl dimethicone crosspolymer, dimethicone PEG10/15 crosspolymer, dimethicone/polyglycerin-3 crosspolymer, lauryl PEG-9 polydimethylsiloxyethyl dimethicone, bis-vinyl dimethicone/bis-isobutyl PPG-20 crosspolymer, bis-vinyl dimethicone/PEG-10 dimethiconecrosspolymer, bis-vinyl dimethicone/PPG-20 crosspolymer, dimethicone/bis-isobutyl PPG-20 crosspolymer, dimethicone/PEG-10 crosspolymer, dimethicone/PEG-10/15 crosspolymer, dimethicone/PEG-15 crosspolymer, dimethicone/PEG-3/PPG-15 crosspolymer, dimethicone/polyglycerin-3 crosspolymer, dimethicone/PEG-20 crosspolymer, PEG-10 dimethicone crosspolymer, PEG-12 dimethicone crosspolymer, PEG-12 dimethicone/PPG-20 crosspolymer, PEG-10 dimethicone/vinyl dimethicone crosspolymer, PEG-10/lauryl dimethicone crosspolymer, PEG-15/lauryl dimethicone crosspolymer, PEG-15/lauryl polydimethylsiloxyethyl dimethiconecrosspolymer, and PEG-10 polydimethylsiloxyethyl dimethicone/bis-vinyl dimethicone crosspolymer, and the like, but not limited thereto. Preferably, the silicon crosspolymer comprising a hydrophilic group may be PEG-10 dimethicone/vinyl dimethicone crosspolymer. In this case, the viscosity (fluidity) of the composition is stabilized at high temperature, thereby the filling property of the composition is improved, and the stability of formulation is remarkably increased.

In one embodiment, the surfactant comprising one hydrophilic group and two hydrophobic groups may be comprised in an amount of 0.05 to 3% by weight, preferably, 0.1 to 2.5% by weight, more preferably, 0.2 to 2% by weight, based on the total weight of the composition. If the surfactant comprising one hydrophilic group and two hydrophobic groups is less than 0.05% by weight, the effect of improving the viscosity and stability may be insignificant. If the surfactant is over 3% by weight, the emulsified particle size and viscosity are not significantly changed, and the stability of formulation and feeling in use may be impaired.

In one embodiment, the silicon crosspolymer comprising a hydrophilic group may be comprised in an amount of 0.05 to 3% by weight, preferably, 0.1 to 2% by weight, more preferably, 0.2 to 1% by weight, based on the total weight of the composition. If the silicon crosspolymer comprising a hydrophilic group is less than 0.05% by weight, the effect of improving the stability and stabilizing emulsion at high temperature may be reduced. If the silicon crosspolymer is over 3% by weight, the stability of formulation and feeling in use may be impaired.

In one embodiment, the weight ratio of the surfactant comprising one hydrophilic group and two hydrophobic groups and the silicon crosspolymer comprising a hydrophilic group may be 1:0.1 to 5, preferably, 1:0.2 to 2.5, more preferably, 1:0.3 to 1.5 (surfactant comprising one hydrophilic group and two hydrophobic groups:silicon crosspolymer comprising a hydrophilic group). If the above two ingredients are combined in the specific weight ratio, the effect of improving viscosity and phase stability of the cosmetic composition at high temperature are remarkably enhanced.

In one embodiment, the wax may be a vegetable wax, an animal wax or a synthetic wax, and the wax may comprise derivatives thereof. For example, the wax may be animal wax such as Shellac wax, lanolin wax or bees wax; vegetable wax such as caster wax, carnauba wax, candelilla wax, ouricury wax, sugarcane wax, retamo wax, rice bran wax, sunflower seed wax or soy wax; petroleum-derived wax such as paraffin wax; mineral wax such as ozocerite, montan wax or ceresin, and the like. In addition, the wax may be polyethylene wax derived from polyethylene, wax produced by chemically synthesizing by esterification or saponification, or the like. Preferably, the wax may be a ceresin. The wax may be comprised in an amount of 2 to 10% by weight, preferably, 2.5 to 9% by weight, more preferably, 3 to 8% by weight, based on the total weight of the composition. If the wax is less than 2% by weight, it is difficult to prepare a desired cosmetic formulation due to insufficient viscosity. If the wax is over 10% by weight, the stability of formulation or feeling in use may be impaired.

In one embodiment, the cosmetic composition may comprise an oil ingredient. The oil ingredient may be a polar oil, a non-polar oil or a mixture thereof. The polar oil may be an ester-based oil, or the like, and the non-polar oil may be a silicon oil or a hydrocarbon oil, or the like, but not limited thereto. In the cosmetic composition of the present invention, the oil ingredient may be a silicon-based oil, an ester-based oil or a mixture thereof. In the preparation of the cosmetic composition of the present invention, it is necessary to select an oil which does not interfere with the formation of a wax matrix. If the polarity of the oil is too low, the wax substrate may be softened. If the polarity is too high, the oil may be not mixed and be separated in the wax matrix. In consideration of the polarity of these oil, the cosmetic composition of the present invention may preferably comprise an ester-based oil. The oil ingredient may be comprised in an amount of 5 to 50% by weight, preferably, 10 to 40% by weight, more preferably, 15 to 35% by weight, based on the total weight of the composition. In addition, the ester-based oil ingredient may be comprised in an amount of 3 to 20% by weight, preferably, 5 to 15% by weight, based on the total weight of the composition. If the oil ingredient is less than 5% by weight, it is difficult to form an oily film on the skin, and to comprise a large amount of aqueous phase, thereby the phase stability may be impaired. If the oil ingredient is over 50% by weight, the viscosity of oil phase may be lowered, thereby the stability of formulation in the molten state may be impaired. In addition, the ratio of aqueous phase may be reduced, thereby the feeling in use such as water-drop effect may be impaired.

In one embodiment, the ester-based oil may be one or more selected from the group consisting of ascorbyl palmitate, ascorbyl linoleate, distearyl malate, benzyl benzoate, benzyl laurate, butylene glycol dicaprylate/dicaprate, butylene glycol diisonanoate, butylene glycol laurate, butylene glycol stearate, butyl isostearate, cetearyl isonanoate, cetearylonanoate, cetyl caprylate, cetyl ethyl hexanoate, cetyl isononanoate, ethyl hexyl caprylate/caprate, ethyl hexyl isononanoate, ethyl hexyl isostearate, ethyl hexyl laurate, hexyl laurate, octyl dodecyl isostearate isopropyl isostearate, isostearyl isononanoate, isostearyl isostearate, isocetyl ethyl hexanoate, neopentyl glycol dicaprate, neopentyl glycol diethylhexanoate, neopentyl glycol diisononanoate, neopentyl glycol diisostearate, pentaerythrityl stearate, pentaerythrityl tetraethyl hexanoate and triethyl hexanoin and the like, but not limited thereto.

In one embodiment, the silicon oil may be one or more selected from the group consisting of cyclomethicone, cyclotetrasiloxane, cyclohexasiloxane, cycloheptasiloxane, decamethyl cyclopentasiloxane, cyclotetrasiloxane, cyclotrisiloxane, dimethicone, capryl dimethicone, caprylyl trimethicone, caprylyl methicone, cetearyl methicone, hexadecyl methicone, hexyl methicone, lauryl methicone, myristyl methicone, phenyl methicone, stearyl methicone, stearyl dimethicone, trifluoropropyl methicone, cetyl dimethicone, polyphenyl methylsiloxane, dimethyl polysiloxane, methylphenyl polysiloxane, methyl trimethicone, diphenylsiloxyphenyl trimethicone, caparylyl methicone and phenyl trimethicone, and the like, but not limited thereto.

In one embodiment, the aqueous phase consisting of the internal phase of the water-in-oil cosmetic composition of the present invention may be 20 to 80% by weight, preferably, 30 to 70% by weight, more preferably, 40 to 60% by weight, based on the total weight of the composition. If the aqueous phase is less than 20% by weight, it is difficult to visually or tactilely exhibit the effect in which the moisture inside bursts out as the membrane of the emulsified particle surrounding moisture breaks (water-drop effect). If the aqueous phase is over 80% by weight, it is difficult to emulsify. The weight ratio of the aqueous phase may be relatively controlled depending on the weight of the surfactant and silicon elastomer to be added when preparing the water-in-oil cosmetic composition.

In one embodiment, the water-in-oil cosmetic composition of the present invention may be prepared in a balm type formulation, a wax stick, a wax cake, a wax pact or a gel type formulation, or the like. Preferably, the water-in-oil cosmetic composition of the present invention may be prepared in a balm type formulation. If it is prepared as the balm-type formulation, the viscosity (fluidity) of the composition is excellent in high temperature preparation process, thereby the filling property and the phase stability are significantly excellent. The water-in-oil cosmetic composition of the present invention may be used for applications such as makeup. In addition, the cosmetic composition may be used for any one application selected from the group consisting of foundation, concealer, makeup base, BB cream, lotion, cream and wax, and the like, and preferably, it may be used as a foundation.

In one embodiment, the balm type formulation may mean a cosmetic composition comprising wax, which is a formulation that exists in a solid state at room temperature and has a fluidity at particular temperature or higher. The balm type formulation may have the hardness of 100 to 5,000, preferably, 200 to 3,000, more preferably, 300 to 1,000 dyne/cm², but not limited thereto. The hardness may be measured for example, in a condition of 2 mm depth at a rate of 2 cm/min using a pin with a diameter of 10ϕ among dedicated adapters \textcircled{3} of Fudoh Reo Meter.

In one embodiment, the water-in-oil cosmetic composition of the present invention may have the viscosity of 1600 to 6000 cps (Centipoise), preferably, 2000 to 5000 cps. The viscosity range may be measured at high temperature, preferably, 60˜100° C., more preferably, 70˜90° C., for example, 80° C. If the viscosity of the composition at high temperature is less than 1600 cps, the formulation stability in the molten state is impaired due to excessive fluidity. If the viscosity is over 6000 cps, the filling property is poor and it is difficult to produce the desired shape due to the excessive viscosity.

In one embodiment, the water-in-oil cosmetic composition of the present invention may have a particle size of 2 to 20 um, preferably, 5˜10 um. The composition of the present invention may control the particle size by adjusting the content of a particular emulsifier. If the particle size is uniformly and stably formed within the above range, the moisturizing feeling (cooling feeling) may be enhanced.

In one embodiment, the water-in-oil cosmetic composition of the present invention may further comprise a commonly used emulsion stabilizer, pigment, ultraviolet blocking agent, moisturizer, thickener, surfactant, oil phase base compound, preservative, anti-oxidant, alcohol, flavor, pH adjusting agent or natural extract, or the like, but not limited thereto. The water-in-oil cosmetic composition of the present invention may not substantially comprise an oil-in-water (O/W) surfactant or may not comprise it at all.

In one embodiment, the present invention provides a balm type water-in-oil cosmetic composition comprising 0.05 to 3% by weight of PEG-30 dipolyhydroxystearate, 0.05 to 3% by weight of PEG-10 dimethicone/vinyldimethicone crosspolymer, 2 to 10% by weight of wax, 3 to 20% by weight of ester-based oil, and 20 to 80% by weight of aqueous phase, based on the total weight of the composition. The water-in-oil cosmetic composition has the excellent filling property, stability at high temperature and long term stability by forming the desirable fluidity due to the combination of the ingredients.

In addition, the present invention provides a method for preparation of a water-in-oil cosmetic composition comprising (S1) dissolving a surfactant comprising one hydrophilic group and two hydrophobic groups, a silicon crosspolymer comprising a hydrophilic group, and wax in oil phase; and (S2) adding aqueous phase to the oil phase prepared in the step (S1) and emulsifying. The water-in-oil cosmetic composition prepared by the method for preparation has the excellent filling property, stability at high temperature and long term stability, by forming a desirable fluidity at high temperature.

In one embodiment, dissolving of the step (S1) may be performed at high temperature, preferably, 65 to 105° C., more preferably, 75 to 95° C. In the step (S2), the aqueous phase ingredients may be previously dissolved at high temperature, preferably, 50 to 90° C., more preferably, 60 to 80° C. before adding the aqueous phase to the oil phase.

### Advantageous Effects

The water-in-oil cosmetic composition in a balm type formulation of the present invention has excellent filling property, stability at high temperature and long term stability, by forming a desirable fluidity in the process of preparation at high temperature. Preferably, the water-in-oil cosmetic composition of the present invention may exhibit excellent water-drop effect due to high content of aqueous phase.

### DETAILED DESCRIPTION OF THE EMBODIMENTS

Hereinafter, to help understanding of the present invention, it will be described in detail by examples, and the like. However, the examples according to the present invention may be modified into various other forms, and the scope of the present invention should not be construed to be limited by the following examples. The examples of the present invention are provided to those skilled in the art to more completely describe the present invention.

### Preparation of Example and Comparative Example

A wax ingredient, an oil phase ingredient, an emulsifier and an ultraviolet blocking agent were added to an oil phase container and heated to 85° C., and then homogenized with a homomixer. After confirming the homogenized state, pigment was added to completely dispersed. A aqueous phase ingredient, an emulsion stabilizer and a preservative were added to the aqueous phase container, and the raw materials were completely dissolved at 70° C. Thereafter, the aqueous phase was added to the oil phase container in which the pigment was dispersed, and then emulsified with a homomixer to prepare water-in-oil foundation emulsion.

TABLE 1

| | | Comparative | | Comparative | Comparative | Comparative |
|---|---|---|---|---|---|---|
| Classification | Ingredient name | example 1 | Example 1 | example 2 | example 3 | example 4 |
| Wax | Ceresin | 4 | 4 | 4 | 4 | 4 |
| ingredient | | | | | | |
| Oil phase | Silicon-based oil | 15.5 | 15 | 15 | 15 | 15.5 |
| ingredient | Ester-based oil | 8 | 8 | 8 | 8 | 8 |
| Emulsifier | PEG-30 | — | 0.5 | — | — | 0.5 |
| | dipolyhydroxy- | | | | | |
| | stearate | | | | | |
| | Cetyl PEG/ | — | — | 0.5 | — | — |
| | PPG-10/1 | | | | | |
| | Dimethicone | | | | | |
| | Polyglyceryl- | — | — | — | 0.5 | — |
| | 4isostearate | | | | | |
| | PEG-10 | 0.5 | 0.5 | 0.5 | 0.5 | — |
| | dimethicone/ | | | | | |
| | vinyldimethicone | | | | | |
| | crosspolymer | | | | | |
| UV blocking | Ethylhexyl- | 7 | 7 | 7 | 7 | 7 |
| agent | methoxycinnamate | | | | | |
| Pigment | Titanium oxide | 9 | 9 | 9 | 9 | 9 |
| | Iron oxide | 1 | 1 | 1 | 1 | 1 |
| Aqueous | Purified water | to 100 | to 100 | to 100 | to 100 | to 100 |
| phase | Butylene glycol | 5 | 5 | 5 | 5 | 5 |
| ingredient | | | | | | |
| Emulsion | Magnesium | 1 | 1 | 1 | 1 | 1 |
| stabilizer | sulfate | | | | | |
| | Disteardimonium | 0.5 | 0.5 | 0.5 | 0.5 | 0.5 |
| | hectorite | | | | | |
| Preservative | 1,2-Hexandiol | 1 | 1 | 1 | 1 | 1 |

### Experimental Example 1: Experiment of Fluidity (Viscosity), Stability at Heat Temperature and Long Term Stability of the Composition

For the Example 1 and Comparative examples 1 to 4, an experiment was conducted to confirm the fluidity (viscosity) and stability at a molding temperature (80° C.). The stability test was conducted by observing the surface condition at high temperature (80° C.) for 4 hours, and the presence or absence of phase separation at 40° C. for 4 weeks.

TABLE 2

| | Compar- | | Compar- | Compar- | Compar- |
|---|---|---|---|---|---|
| | ative | | ative | ative | ative |
| | exam- | Exam- | exam- | exam- | exam- |
| | ple 1 | ple 1 | ple 2 | ple 3 | ple 4 |
| Fluidity | 8200 | 2400 | 6400 | 7500 | 1500 |
| (viscosity | | | | | |
| cPs)* | | | | | |
| High | X | ◯ | Δ | X | Δ |
| temperature | | | | | |
| stability | | | | | |
| (4 hours) | | | | | |
| Long term | ◯ | ◯ | ◯ | ◯ | Δ |
| stability | | | | | |
| (4 weeks) | | | | | |

*Viscosity measurement: 80° C., 30 rpm, 1 min LVT Brookfield viscometer, spindle No. 4 used

In case of the Example 1 and Comparative example 4, it was confirmed that the fluidity was greatly improved, and filling was smoothly done to the edge of the dish. In particular, Example 1 was confirmed that the high temperature stability and long term stability for 4 weeks was significantly improved.

### Experimental Example 2: Experiment of Fluidity and Stability Change Depending on the Content of Surfactant

The change in fluidity, behavior of emulsified particles and stability depending on the content of the emulsifier were comparatively tested, based on the Example 1 with excellent fluidity (viscosity) and stability.

TABLE 3

| | | Comparative | | | | Comparative |
|---|---|---|---|---|---|---|
| Classification | Ingredient name | example 1 | Example 1 | Example 2 | Example 3 | example 2 |
| Wax | Ceresin | 4 | 4 | 4 | 4 | 4 |
| ingredient | | | | | | |
| Oil phase | Silicon-based oil | 15.5 | 15 | 15.25 | 14.5 | 15 |
| ingredient | Ester-based oil | 8 | 8 | 8 | 8 | 8 |
| Emulsifier | PEG-30 | — | 0.50 | 0.25 | 1.00 | — |
| | dipolyhydroxy- | | | | | |
| | stearate | | | | | |
| | Cetyl PEG/ | — | — | — | — | 0.5 |
| | PPG-10/1 | | | | | |
| | Dimethicone | | | | | |
| | PEG-10 | 0.5 | 0.5 | 0.5 | 0.5 | 0.5 |
| | dimethicone/ | | | | | |
| | vinyldimethicone | | | | | |
| | crosspolymer | | | | | |
| UV blocking | Ethylhexyl- | 7 | 7 | 7 | 7 | 7 |
| agent | methoxycinnamate | | | | | |
| Pigment | Titanium oxide | 9 | 9 | 9 | 9 | 9 |
| | Iron oxide | 1 | 1 | 1 | 1 | 1 |
| Aqueous | Purified water | to 100 | to 100 | to 100 | to 100 | to 100 |
| phase | Butylene glycol | 5 | 5 | 5 | 5 | 5 |
| ingredient | | | | | | |
| Emulsion | Magnesium sulfate | 1 | 1 | 1 | 1 | 1 |
| stabilizer | Disteardimonium | 0.5 | 0.5 | 0.5 | 0.5 | 0.5 |
| | hectorite | | | | | |
| Preservative | 1,2-Hexandiol | 1 | 1 | 1 | 1 | 1 |

The method for preparation of Examples 2 and 3 was conducted as same as the method for preparation of the example and comparative examples. In addition, the fluidity and stability experiment were conducted in the same manner as the Experimental example 1.

TABLE 4

| | Compar- | | | | Compar- |
|---|---|---|---|---|---|
| | ative | | | | ative |
| | exam- | Exam- | Exam- | Exam- | exam- |
| | ple 1 | ple 1 | ple 2 | ple 3 | ple 2 |
| Fluidity | 8200 | 2400 | 3900 | 2300 | 6400 |
| (viscosity)* | | | | | |
| High | X | ◯ | ◯ | ◯ | Δ |
| temperature | | | | | |
| stability | | | | | |
| (4 hours) | | | | | |
| Long term | ◯ | ◯ | ◯ | ◯ | ◯ |
| stability | | | | | |
| (4 weeks) | | | | | |
| Emulsified | Hetero- | Homo- | Homo- | Homo- | Homo- |
| particle | geneous | geneous | geneous | geneous | geneous |
| size ** | | | | | |

*Viscosity measurement: 80° C., 30 rpm, 1 min LVT Brookfield viscometer, spindle No. 4 used
** Observing the emulsified particle size at high temperature (80° C.) by a magnifying glass in 4 hours

In Examples 1 to 3, it was confirmed that the emulsified particles became homogeneous and the fluidity was improved as the content of the surfactant (PEG-30 dipolyhydroxystearate) increased. If the surfactant was in a particular amount or more, the change of the emulsified particle size and fluidity were not great according to the increase in the amount of surfactant. In addition, in Comparative example 1, the emulsified particles were heterogeneously formed, and the aqueous phase was separated outside of the formulation at high temperature. In addition, in Comparative example 2, the stability at high temperature was lowered than Examples 1 to 3.

From the above experimental result, it was found that the formulation was stabilized without increase of viscosity even though aqueous phase was largely formed by comprising a combination of specific emulsifiers. In addition, the moisturizing feeling (cooling feeling) was improved by stabilizing emulsified particles over a particular size.

### Experimental Example 3: Emulsified Particle Size and Sensory Evaluation

For Example 1 and Comparative examples 2 and 3, the emulsified particle size was analyzed by instrument, and sensory evaluation was conducted to evaluate the moisturizing feeling (cooling feeling).

TABLE 5

| | | Compar- | Compar- |
|---|---|---|---|
| | | ative | ative |
| | Exam- | exam- | exam- |
| | ple 1 | ple 2 | ple 3 |
| Emulsified particle size* | 6.21±1.54 | 4.04 ± 0.66 | 7.10 ± 3.98 |
| Cooling feeling** | Δ3.0 | Δ2.9 | Δ3.0 |
| Moisturizing feeling | 4.85 | 4.00 | 4.25 |
| sensory evaluation*** | | | |
| Overall product | 4.5 | 3.9 | 4.1 |
| satisfaction*** | | | |

*Observing the formulation surface with a magnifying glass (×300)
**Temperature difference before and after skin application (20 consumers in 20s to 30s)
***Consumer evaluation on a 5-point scale (20 consumers in 20s to 30s)

Example 1 showed the excellent water-drop effect and cooling feeling as the emulsified particles were formed in a particular size. In addition, the moisturizing feeling sense and overall product satisfaction were significantly excellent, compared to Comparative examples 2 and 3.

1. A method for preparation of a water-in-oil cosmetic composition, comprising:
(S1) dissolving a surfactant comprising one hydrophilic group and two hydrophobic groups, a silicon crosspolymer comprising a hydrophilic group, and wax in oil phase; and
(S2) adding aqueous phase to the oil phase prepared in the step (S1) and emulsifying.

2. The method according to claim 1, wherein the wax is comprised in an amount of 2 to 10% by weight based on the total weight of the composition.

3. The method according to claim 1, wherein the water-in-oil cosmetic composition is a balm type.

4. The method according to claim 1, wherein the surfactant comprising one hydrophilic group and two hydrophobic groups is a surfactant having a structure in which one hydrophilic group forms a polar head, and a chain saturated hydrocarbon having 16˜24 carbon atoms or derivatives thereof are extended to both sides of the polar head.

5. The method according to claim 1, wherein the surfactant comprising one hydrophilic group and two hydrophobic groups is comprised in an amount of 0.05 to 3% by weight based on the total weight of the composition.

6. The method according to claim 1, wherein the silicon crosspolymer comprising a hydrophilic group is comprised in an amount of 0.05 to 3% by weight based on the total weight of the composition.

7. The method according to claim 1, wherein the cosmetic composition comprises ester-based oil.

8. The method according to claim 7, wherein the ester-based oil is comprised in an amount of 3 to 20% by weight based on the total weight of the composition.

9. The method according to claim 1, wherein the cosmetic composition comprises aqueous phase in an amount of 20 to 80% by weight based on the total weight of the composition.

10. The method according to claim 1, wherein the viscosity of the cosmetic composition is 1600 to 6000 cps.

11. The method according to claim 1, wherein the size of the emulsified particle in the cosmetic composition is 2 to 20 um.

12. A method for preparation of a balm type water-in-oil cosmetic composition, comprising:
(S1) dissolving 0.05 to 3% by weight of PEG-30 dipolyhydroxystearate, 0.05 to 3% by weight of PEG-10 dimethicone/vinyldimethicone crosspolymer, 2 to 10% by weight of wax, and 3 to 20% by weight of ester-based oil, based on the total weight of the composition, in oil phase; and
(S2) adding 20 to 80% by weight of aqueous phase, based on the total weight of the composition, to the oil phase prepared in the step (S1) and emulsifying.
